(19) United States
(12) Patent Application Publication
(10) Pub. No.: US 2015/0173638 A1
(43) Pub. Date: Jun. 25, 2015

(54) DIRECT COMPARISON OF MULTIPLE HEALTHCARE WAVES

(71) Applicant: CERNER INNOVATION, INC., Kansas City, KS (US)

(72) Inventors: Damian Kiesling, Olathe, KS (US); Bryce Shaffter, Kansas City, MO (US); Cristin Marker, Blue Springs, MO (US)

(21) Appl. No.: 14/567,881

(22) Filed: Dec. 11, 2014

Related U.S. Application Data
(60) Provisional application No. 61/920,375, filed on Dec. 23, 2013.

(51) Int. Cl. A61B 5/044; A61B 5/021; A61B 5/0476; G06F 19/00; A61B 5/00; G06F 3/0481; G09G 5/14; A61B 5/0444; A61B 5/08

(52) U.S. Cl. CPC A61B 5/044; A61B 5/0444; A61B 5/021; A61B 5/0476; A61B 5/0816; A61B 5/4356; G06F 3/0481; G09G 5/14; G06F 19/3406; G09G 2340/12

(57) ABSTRACT

Methods and systems are directed to the evaluation of a particular healthcare activity by comparing the plurality of waves of the healthcare activity from two or more of the same healthcare activity. This includes the electrocardiogram (ECG or EKG) and its use in comparing two or more ECGs of a patient. Two or more different ECG tracings are overlaid in order to compare them directly. Physicians also have the ability to time-shift one or more of the waves forward or backward in time so that relevant points on the waves line up and better correlate with the other corresponding waves. The ECG tracings and the waves are displayed on a graphical user interface (GUI).

### CROSS-REFERENCE TO RELATED APPLICATIONS

This application claims priority to U.S. Provisional Patent Application having Ser. No. 61/920,375, filed on Dec. 23, 2013, which is hereby incorporated by reference in its entirety.

### BACKGROUND

Graphically illustrated waves are utilized throughout the healthcare field as diagnostic and monitoring tools for various medical conditions and functions. For example, waves can be used to monitor or evaluate blood pressure, heart morphology and function, fetal heart beat, brain function, respiratory function, maternal uterine activity, and the like (hereinafter “healthcare activities”). In general, there is a “normal” wave pattern that indicates no health concern to the patient. In contrast, “abnormal” wave patterns indicate some type of medical problem or complication concerning the patient. Thus, these waves (hereinafter “healthcare waves”), play a large role in diagnosing and monitoring a patient's health. The healthcare waves are usually displayed as a plurality of waves, also known as a strip or tracing.

One example of a healthcare activity is electrocardiography, which is one of the simplest and fastest procedures used to evaluate the heart. It is a procedure in which the electrical activity of the heart is measured over a period of time. The electrical activity of the heart is detected by electrodes attached to the surface of the skin that are placed on each limb and across the chest of the patient. An electrocardiogram (ECG or EKG) is the recording produced by this noninvasive procedure. The ECG is used to measure the heart's conduction system as it picks up electrical impulses generated by the depolarization of the cardiac tissue and translates this into ECG waves (or an ECG tracing) that is made up of a plurality of waves that make up individual heartbeats that can be evaluated by the treating physician. The waves of the tracing are then used to measure the rate and regularity of heartbeats, the size and position of the heart chambers, the presence of any damage to the heart, and the effects of drugs or devices used to regulate the heart, such as a pacemaker.

### BRIEF SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter. The present invention is defined by the claims.

Embodiments of the present invention relate generally to the evaluation of a particular healthcare activity by comparing the plurality of waves of the healthcare activity from two or more of the same healthcare activity. One example is the electrocardiogram (ECG or EKG) and its use in comparing two or more ECGs of a patient. Currently, physicians have to view two or more ECGs of a patient side-by-side, either horizontally or vertically. Physicians could use a way to directly compare the latest ECG tracing to previous ECG tracing(s) to aid in their assessment of the patient. One embodiment of the present invention relates to the ability to overlay two or more different ECG tracings in order to compare them directly. This concept allows ECG tracings to be overlaid directly on top of one another allowing a physician a clear view of the differences between the two or more tracings as a whole or between two or more corresponding waves. In another embodiment, physicians also have the ability to time-shift one or more of the waves forward or backward in time so that relevant points on the waves line up and better correlate with the other corresponding waves. In an embodiment, the ECG tracings and the waves are displayed on a graphical user interface (GUI).

### BRIEF DESCRIPTION OF THE FIGURES

Embodiments are described in detail below with reference to the attached figures, wherein:

FIG. 1 is a diagram depicting an exemplary computing environment suitable for use in accordance with an embodiment of the invention;

FIGS. 2A and 2B depict an ECG tracing from a patient with a normal heartbeat with a close-up view of one of the heartbeats;

FIG. 3 is a block diagram depicting a method for displaying overlapping ECGs of a patient on a GUI;

FIG. 4 is a block diagram depicting a method for overlapping tracings of healthcare waves from the same type of healthcare activity; and

FIG. 5 depicts a GUI in which two different ECG tracings are displayed with each displayed in a different color.

### DETAILED DESCRIPTION

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

As one skilled in the art will appreciate, embodiments of our invention may be embodied as, among other things: a set of instructions embodied on one or more non-transitory computer-readable media, or a graphical user interface (GUI) embodied on one or more non-transitory computer-readable media. Accordingly, the embodiments may take the form of a hardware embodiment, a software embodiment, or an embodiment combining software and hardware. In one embodiment, the invention takes the form of a computer-program product that includes computer-usable instruction embodied on one or more non-transitory computer-readable media.

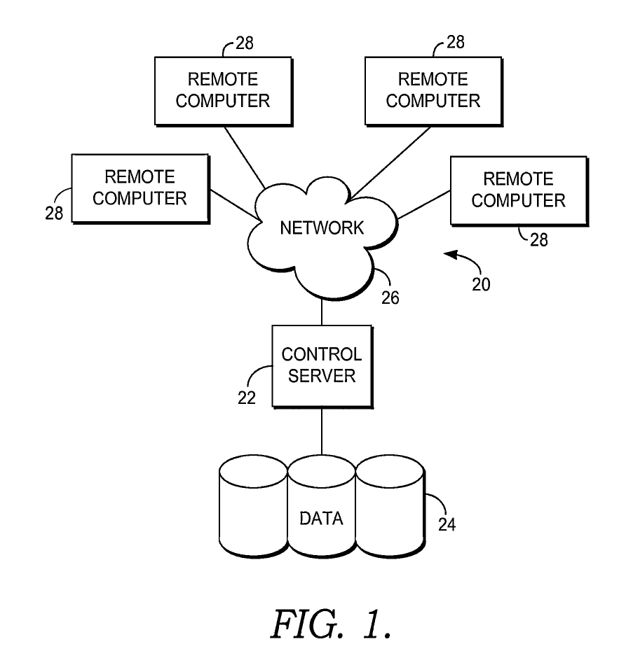

An exemplary operating environment suitable for use in implementing embodiments of the present invention is described below. Referring to the drawings in general, and initially to FIG. 1 in particular, an exemplary computing system environment, a medical information computing system environment, with which embodiments of the present invention may be implemented is illustrated and designated generally as reference numeral 20. It will be understood and appreciated by those of ordinary skill in the art that the illustrated medical information computing system environment 20 is merely an example of one suitable computing environment and is not intended to suggest any limitation as to the scope of use or functionality of the invention. Neither should the medical information computing system environment 20 be interpreted as having any dependency or requirement relating to any single component or combination of components illustrated therein.

The present invention may be operational with numerous other general purpose or special purpose computing system environments or configurations. Examples of well-known computing systems, environments, and/or configurations that may be suitable for use with the present invention include, by way of example only, personal computers, server computers, hand-held or laptop devices, multiprocessor systems, microprocessor-based systems, set top boxes, programmable consumer electronics, personal digital assistants, network PCs, minicomputers, mainframe computers, distributed computing environments that include any of the above-mentioned systems or devices and the like.

The present invention may be described in the general context of computer-executable instructions, such as program modules, being executed by a computer. Generally, program modules include, but are not limited to, routines, programs, objects, components, and data structures that perform particular tasks or implement particular abstract data types. The present invention may also be practiced in distributed computing environments where tasks are performed by remote processing devices that are linked through a communications network. In a distributed computing environment, program modules may be located in association with local and/or remote computer storage media including, by way of example only, memory storage devices.

With continued reference to FIG. 1, the exemplary medical information computing system environment 20 includes a general purpose computing device in the form of a control server 22. Components of the control server 22 may include, without limitation, a processing unit, internal system memory, and a suitable system bus for coupling various system components, including database cluster 24, with the control server 22. The system bus may be any of several types of bus structures, including a memory bus or memory controller, a peripheral bus, and a local bus, using any of a variety of bus architectures. By way of example, and not limitation, such architectures include Industry Standard Architecture (ISA) bus, Micro Channel Architecture (MCA) bus, Enhanced ISA (EISA) bus, Video Electronic Standards Association (VESA) local bus and Peripheral Component Interconnect (PCI) bus, also known as Mezzanine bus.

The control server 22 typically includes therein, or has access to, a variety of computer-readable media and/or communications media, for instance, database cluster 24. Computer-readable media can be any available media that may be accessed by server 22 and includes volatile and nonvolatile media as well as removable and non-removable media. But computer-readable media is exclusive of communications media.

By way of example, and not limitation, computer-readable media include computer storage media and computer storage devices. Computer storage media may include, without limitation, volatile and nonvolatile media as well as removable and non-removable media implemented in any method or technology for storage of information, such as computer-readable instructions, data structures, program modules or other data. In this regard, non-transitory computer storage media may include, but is not limited to, RAM, ROM, EEPROM, flash memory or other memory technology, CD-ROM, digital versatile disks (DVDs) or other optical disk storage, magnetic cassettes, magnetic tape, magnetic disk storage or other magnetic storage device or any other medium which can be used to store the desired information and which may be accessed by the control server 22.

By way of example, and not limitation, communication media includes wired media such as a wired network or direct-wired connection, and wireless media such as acoustic, RF, infrared and other wireless media. And communications media is inclusive of signals, carrier waves and the like.

The computer storage media discussed above and illustrated in FIG. 1, including database cluster 24, provide storage of computer-readable instructions, data structures, program modules and other data for the control server 22. The control server 22 may operate in a computer network 26 using logical connections to one or more remote computers 28. Remote computers 28 may be located at a variety of locations in a medical or research environment, for example, but not limited to, clinical laboratories (e.g., molecular diagnostic laboratories), hospitals and other inpatient settings, veterinary environments, ambulatory settings, medical billing and financial offices, hospital administration settings, home health care environments and clinicians' offices. Clinicians may include, but are not limited to, a treating physician or physicians, specialists such as surgeons, radiologists, cardiologists, and oncologists, emergency medical technicians, physicians' assistants, nurse practitioners, nurses, nurses' aides, pharmacists, dieticians, microbiologists, laboratory experts, laboratory technologists, genetic counselors, researchers, veterinarians, students and the like. The remote computers 28 may also be physically located in non-traditional medical care environments so that the entire health care community may be capable of integration on the network. The remote computers 28 may be personal computers, servers, routers, network PCs, peer devices, other common network nodes or the like, and may include some or all of the elements described above in relation to the control server 22.

Exemplary computer networks 26 may include, without limitation, local area networks (LANs) and/or wide area networks (WANs). Such networking environments are commonplace in offices, enterprise-wide computer networks, intranets and the Internet. When utilized in a WAN networking environment, the control server 22 may include a modem or other means for establishing communications over the WAN, such as the Internet. In a networked environment, program modules or portions thereof may be stored in association with the control server 22, the database cluster 24 or any of the remote computers 28. For example, and not by way of limitation, various application programs may reside on the memory associated with any one or more of the remote computers 28. It will be appreciated that the network connections shown are exemplary and other means of establishing a communications link between the computers (e.g., control server 22 and remote computers 28) may be utilized.

In operation, a clinician may enter commands and information into the control server 22 or convey the commands and information to the control server 22 via one or more of the remote computers 28 through input devices, such as a keyboard, a pointing device (commonly referred to as a mouse), a trackball or a touch pad. Other input devices may include, without limitation, microphones, satellite dishes, scanners or the like. Commands and information may also be sent directly from a remote healthcare device to the control server 22. In addition to a monitor, the control server 22 and/or remote computers 28 may include other peripheral output devices, such as speakers and a printer.

Although many other internal components of the control server 22 and the remote computers 28 are not shown, it is appreciated that such components and their interconnection are well known. Accordingly, additional details concerning the internal construction of the control server 22 and the remote computers 28 are not further disclosed herein.

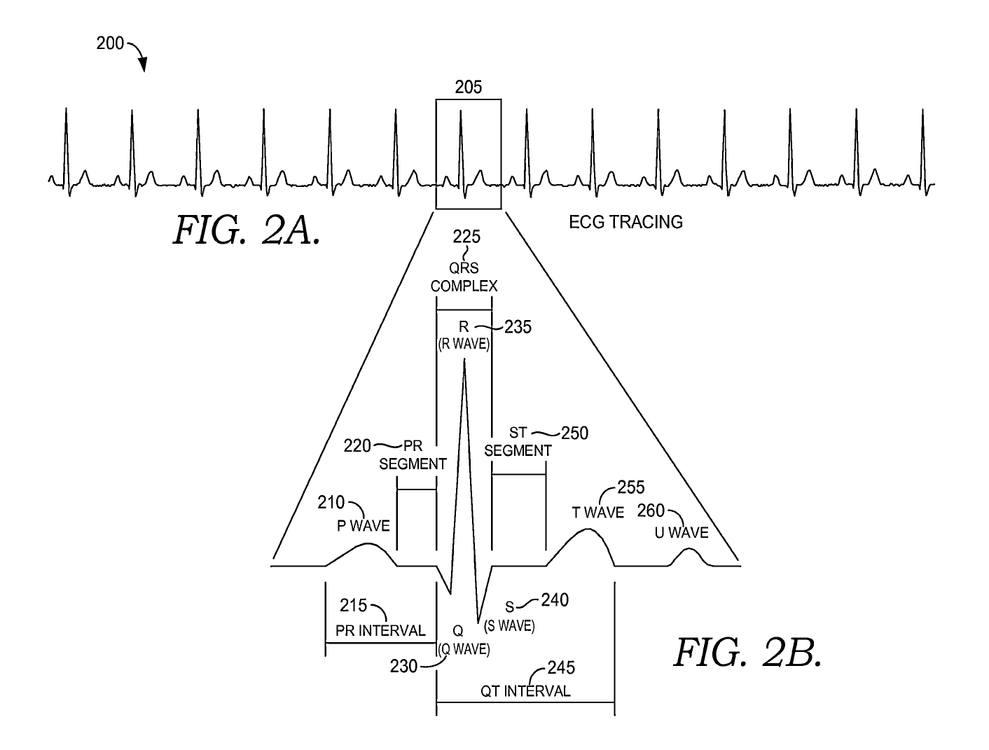

Embodiments of the present invention are directed to non-transitory computer-readable storage media and a GUI for facilitating the overlap of two or more tracings of healthcare waves of a particular healthcare activity. One example is an ECG tracing, or an ECG strip, which is a graphical output of a plurality of heart beats. An ECG tracing 200 is illustrated in FIG. 2A, while a single heartbeat 205 is illustrated in FIG. 2B. As illustrated in FIG. 2B, a single heartbeat comprises a P wave 210, a PR Interval 215, a PR Segment 220, a QRS Complex 225 (i.e. QRS Interval; Q wave 230, R wave 235, S wave 240), a QT Interval 245, a ST Segment 250, a T wave 255, and a U wave 260.

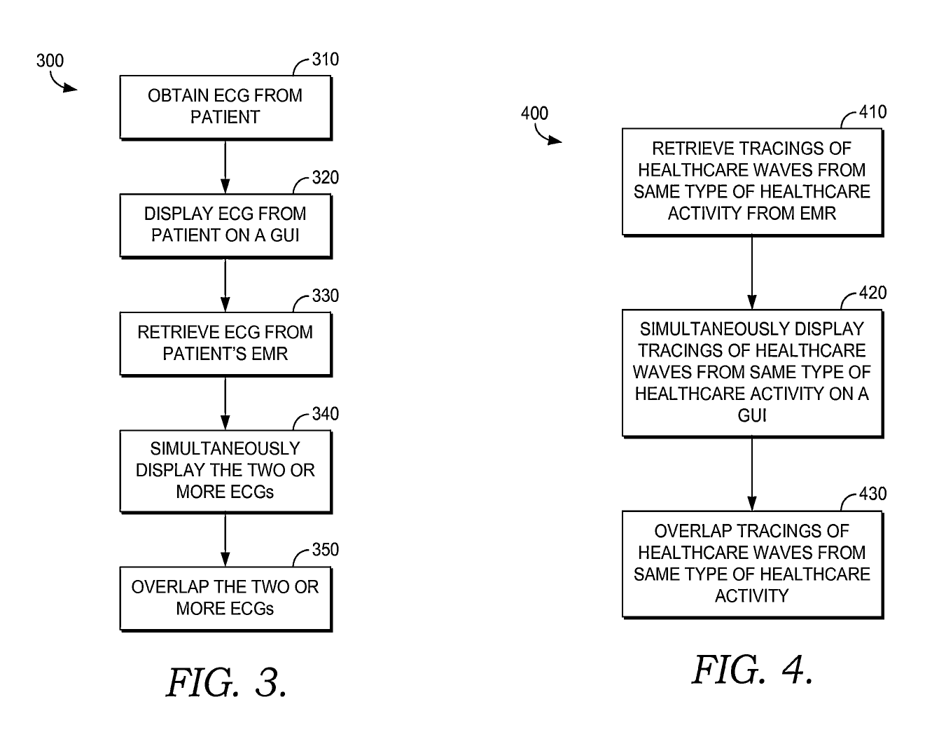

As illustrated in FIG. 3, in one embodiment, there is a non-transitory computer-readable storage media having computer-executable instructions embodied thereon that, when executed, implement a method in a computerized health care environment for simultaneously displaying two or more electrocardiogram (ECG) tracings from a patient, the method 300 comprising: obtaining a current ECG tracing from the patient at a current time 310; displaying the current ECG tracing from the patient on a graphical user interface (GUI) 320; retrieving one or more ECG tracings from the patient's electronic medical record (EMR) that were obtained at a later time than the current time 330; simultaneously displaying the current ECG tracing with the one or more ECG tracings obtained at the later time than the current time on the GUI 340; and overlapping the current ECG tracing with the one or more ECG tracings obtained at the later time such that at least one wave from the current ECG tracing overlaps with at least one corresponding wave from the one or more ECG tracings obtained at a later time than the current time 350.

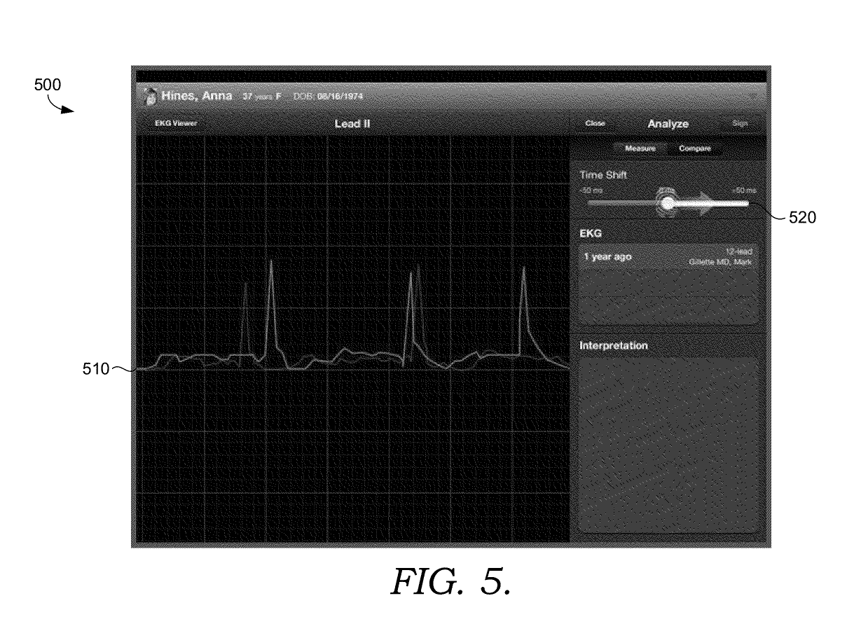

As illustrated in FIG. 4, in another embodiment, there is non-transitory computer-readable storage media having computer-executable instructions embodied thereon that, when executed, implement a method in a computerized health care environment for simultaneously displaying two or more of the same type of healthcare activity from a patient, the method 400 comprising: retrieving two or more tracings of healthcare waves from the same type of healthcare activity, wherein the two or more tracings of the healthcare waves are retrieved from the patient's electronic medical record (EMR) with each obtained at a different time 410; simultaneously displaying the two or more tracings of the healthcare waves from the same type of the healthcare activity, wherein the two or more tracings of the healthcare waves from the patient's EMR are displayed on a graphical user interface (GUI) 420; and overlapping the two or more tracings of the healthcare waves from the same type of the healthcare activity, wherein at least two corresponding waves from the two or more tracings of the healthcare waves overlap 430.

In a further embodiment, there is a graphical user interface (GUI) stored on one or more non-transitory computer-readable media executable by a computing device, the GUI comprising: a first display area on the GUI that represents a patient's ECG tracing at a current time; a second display area on the GUI that represents the patient's one or more ECG tracing(s) obtained at a later time than the current time; and an overlapping display area in which the first display area and the second display area overlap with each other on the GUI, wherein the first and second display areas overlap in such a way that a wave from the current ECG tracing overlaps with a corresponding wave from the one or more ECG tracing(s) obtained at a later time than the current time.

In one embodiment, each healthcare tracing of a plurality of healthcare tracings can be of a different color when displayed on the GUI. This is illustrated in FIG. 5. In one embodiment, each ECG tracing of a plurality of ECG tracings 510 can be of a different color when displayed on the GUI 500. This allows the physician to easily distinguish the various ECG tracings from each other while comparing them. In another embodiment, physicians also have the ability to time-shift 520 one or more of the waves forward or backward in time so that relevant points on the waves line up and better correlate with the other corresponding waves.

It will be apparent to those skilled in the art that various modifications and variations can be made in the practice of the present method and in construction and use of the present technology departing from the scope or spirit of the invention. Other embodiments of the invention will be apparent to those skilled in the art from consideration of the specification and practice of the invention.

What is claimed:

1. Non-transitory computer-readable storage media having computer-executable instructions embodied thereon that, when executed, implement a method in a computerized health care environment for simultaneously displaying two or more electrocardiogram (ECG) tracings from a patient, the method comprising:
obtaining a current ECG tracing from the patient at a current time;
displaying the current ECG tracing from the patient on a graphical user interface (GUI);
retrieving one or more ECG tracings from the patient's electronic medical record (EMR) that were obtained at a different time than the current time;
simultaneously displaying the current ECG tracing with the one or more ECG tracings obtained at the different time than the current time on the GUI; and
overlapping the current ECG tracing with the one or more ECG tracings obtained at the different time such that at least one wave from the current ECG tracing overlaps with at least one corresponding wave from the one or more ECG tracings obtained at a different time than the current time.

2. The non-transitory computer-readable storage media of claim 1, wherein one or more waves displayed on any of the ECG tracings can be time-shifted by the user such that one or more of the waves can shift forward or backward in time, wherein time is represented along a vertical axis of the ECG tracings displayed on the GUI.

3. The non-transitory computer-readable storage media of claim 1, wherein each ECG tracing is a plurality of heart beats.

4. The non-transitory computer-readable storage media of claim 3, wherein each heart beat comprises a P wave, a QRS complex, a T wave and a U wave.

5. The non-transitory computer-readable storage media of claim 1, wherein the one or more ECG tracings are of different colors from the current ECG tracing when displayed.

6. Non-transitory computer-readable storage media having computer-executable instructions embodied thereon that, when executed, implement a method in a computerized health care environment for simultaneously displaying two or more of the same type of healthcare activity from a patient, the method comprising:
retrieving two or more tracings of healthcare waves from the same type of healthcare activity, wherein the two or more tracings of the healthcare waves are retrieved from the patient's electronic medical record (EMR) with each obtained at a different time;
simultaneously displaying the two or more tracings of the healthcare waves from the same type of the healthcare activity, wherein the two or more tracings of the healthcare waves from the patient's EMR are displayed on a graphical user interface (GUI); and
overlapping the two or more tracings of the healthcare waves from the same type of the healthcare activity, wherein at least two corresponding waves from the two or more tracings of the healthcare waves overlap.

7. The non-transitory computer-readable storage media of claim 6, wherein one or more waves displayed on any of the healthcare waves can be time-shifted by the user such that the one or more of the waves can shift forward or backward in time, wherein time is represented along a vertical axis of the healthcare activity displayed on the GUI.

8. The non-transitory computer-readable storage media of claim 6, wherein the healthcare activity is an ECG of the patient.

9. The non-transitory computer-readable storage media of claim 6, wherein the healthcare activity is a blood pressure of the patient.

10. The non-transitory computer-readable storage media of claim 6, wherein the healthcare activity is a fetal heart beat of a pregnant patient.

11. The non-transitory computer-readable storage media of claim 6, wherein the healthcare activity is a brain function of the patient.

12. The non-transitory computer-readable storage media of claim 6, wherein the healthcare activity is a respiratory function of the patient.

13. The non-transitory computer-readable storage media of claim 6, wherein the healthcare activity is a maternal uterine activity of a pregnant patient.

14. A graphical user interface (GUI) stored on one or more non-transitory computer-readable media executable by a computing device, the GUI comprising:
a first display area on the GUI that represents a patient's ECG tracing at a current time;
a second display area on the GUI that represents the patient's one or more ECG tracing(s) obtained at a different time than the current time; and
an overlapping display area in which the first display area and the second display area overlap with each other on the GUI, wherein the first and second display areas overlap in such a way that a wave from a current ECG tracing overlaps with a corresponding wave from the one or more ECG tracing(s) obtained at the different time than the current time.

15. The GUI of claim 14, wherein the patient's ECG tracing at the current time and the patient's one or more ECG tracing(s) obtained at the different time than the current time are displayed as different colors on the GUI.

16. The GUI of claim 14, wherein each ECG tracing is a plurality of heart beats.

17. The GUI of claim 14, wherein each heart beat comprises a P wave, a QRS complex, a T wave and a U wave.

18. The GUI of claim 14, wherein one or more of displayed waves can be time-shifted by the user such that the one or more of the displayed waves can shift forward or backward in time, wherein time is represented along a vertical axis of the healthcare activity displayed on the GUI.
